(19) United States
(12) Patent Application Publication
(10) Pub. No.: US 2018/0036395 A1
(43) Pub. Date: Feb. 8, 2018

(54) DOSAGE AND ADMINISTRATION OF MONOSPECIFIC AND BISPECIFIC ANTI-IGR-1R AND ANTI-ERBB3 ANTIBODIES

(71) Applicant: Merrimack Pharmaceuticals, Inc., Cambridge, MA (US)

(72) Inventors: Alexey Alexandrovich LUGOVSKOY, Belmont, MA (US); Jason BAUM, Needham, MA (US); Sharlene ADAMS, Waltham, MA (US); Bryan JOHNSON, Natick, MA (US); Jian TANG, Newton, MA (US)

(21) Appl. No.: 15/597,781

(22) Filed: May 17, 2017

Related U.S. Application Data
(63) Continuation of application No. 14/388,330, filed on Sep. 26, 2014, now abandoned.
(60) Provisional application No. 61/723,582, filed on Nov. 7, 2012.
(60) Provisional application No. 61/619,258, filed on Apr. 2, 2012.

(51) Int. Cl. A61K 39/00; A61K 31/337; A61K 31/4196; A61K 31/138; C07K 16/28; A61K 31/436; A61K 31/565; A61K 31/506; A61K 31/4745; A61K 31/44; A61K 31/437; C07K 16/40; A61K 31/5685

(52) U.S. Cl. CPC A61K 39/0011; C07K 16/40; A61K 31/337; A61K 31/4196; A61K 31/138; C07K 16/2863; A61K 31/5685; A61K 31/565; A61K 31/506; A61K 31/4745; A61K 31/44; A61K 31/437; A61K 31/436; A61K 2039/505; A61K 2039/545; C07K 2317/31

(57) ABSTRACT

Provided are methods for the administration of therapeutic bispecific anti-IGF-1R and anti-ErbB3 antibodies, either alone or in combination with other anti-cancer therapeutics.

### RELATED APPLICATIONS

This application is a continuation of U.S. patent application Ser. No. 14/388,330, filed Sep. 26, 2014, which is a 35 U.S.C. 371 national filing of International Application No. PCT/US2013/035013, filed Apr. 2, 2013, which claims priority to U.S. Provisional Application Ser. No. 61/723,582, filed Nov. 7, 2012, and U.S. Provisional Application Ser. No. 61/619,258, filed Apr. 2, 2012. The contents of the aforementioned applications are hereby incorporated by reference.

### SEQUENCE LISTING

The instant application contains a Sequence Listing which has been submitted via EFS-Web and is hereby incorporated by reference in its entirety. Said ASCII copy, created on May 17, 2017, is named MMJ_064USCN_Sequence_Listing.txt and is 645,073 bytes in size.

### FIELD OF THE INVENTION

Provided are methods for the administration of therapeutic bispecific anti-IGF-1R and anti-ErbB3 antibodies, either alone or in combination with other anti-cancer therapeutics.

### BACKGROUND OF THE INVENTION

Tumor cells express receptors for growth factors and cytokines that stimulate proliferation of the cells. Antibodies to such receptors can be effective in blocking the stimulation of cell proliferation mediated by growth factors and cytokines and can thereby inhibit tumor cell proliferation and tumor growth. Commercially available therapeutic antibodies that target receptors on cancer cells include, for example, trastuzumab which targets the HER2 receptor (also known as ErbB2) for the treatment of breast cancer, and cetuximab which targets the epidermal growth factor receptor (EGFR, also known as HER1 or ErbB1) for the treatment of colorectal cancer and head and neck cancer.

Monoclonal antibodies have significantly advanced our ability to treat cancers, yet clinical studies have shown that many patients do not adequately respond to monospecific therapy. This is in part due to the multigenic nature of cancers, where cancer cells rely on multiple and often redundant pathways for proliferation. Bi- or multi-specific antibodies capable of blocking multiple growth and survival pathways at once have a potential to better meet the challenge of blocking cancer growth, and indeed many of them are advancing in clinical development. In addition, in the treatment of cancers, the co-administration of pluralities of anti-cancer drugs (combination therapy) often provides better treatment outcomes than monotherapy.

### SUMMARY

A number of isolated polyvalent bispecific antibodies (PBA), are described in co-pending U.S. patent application 61/558,192. These antibodies bind specifically to human IGF-1R and to human ErbB3. These proteins are potent inhibitors of tumor cell proliferation and of signal transduction through either or both of IGF-1R and ErbB3.

Monotherapy with a bispecific anti-IGF-1R and anti-ErbB3 antibody suppresses tumor growth in a dose-dependent manner in in vivo xenograft models of a variety of cancers including pancreatic cancer, renal cell carcinoma, Ewing's sarcoma, non-small cell lung cancer, gastrointestinal neuroendocrine cancer, estrogen receptor positive locally advanced or metastatic cancer, ovarian cancer, colorectal cancer, endometrial cancer, or glioblastoma. It has now been discovered that co-administration of a bispecific anti-IGF-1R and anti-ErbB3 antibody with one or more additional anti-cancer agents, such as everolimus, capecitabine, or XL147, exhibits therapeutic synergy.

Accordingly, provided are methods for the treatment of a cancer in a human patient by administering an effective amount of a bispecific anti-IGF-1R and anti-ErbB3 antibody to the patient, where the patient is given a single loading dose of at least 10 mg/kg of the bispecific antibody followed administration of one or more maintenance doses given at intervals. The intervals between doses are intervals of at least three days. In some embodiments, the intervals are every seven days, every fourteen days or every twenty-one days.

The doses administered may range from 1 mg/kg to 60 mg/kg of the bispecific antibody. In some embodiments, the loading dose is greater than the maintenance dose. The loading dose may from 12 mg/kg to 20 mg/kg, from 20 mg/kg to 40 mg/kg, or from 40 mg/kg to 60 mg/kg. In some embodiments the loading dose is about 12 mg/kg, 20 mg/kg, 40 mg/kg, or 60 mg/kg. In other embodiments the maintenance dose is about 6 mg/kg, 12 mg/kg, 20 mg/kg, 30 mg/kg, 40 mg/kg, 50 mg/kg or 60 mg/kg.

In some embodiments the patient has a pancreatic cancer, renal cell carcinoma, hepatocellular carcinoma, Ewing's sarcoma, non-small cell lung cancer, gastrointestinal neuroendocrine cancer, estrogen receptor- or progesterone receptor-positive locally advanced or metastatic breast cancer, ovarian cancer, triple negative breast cancer, colorectal cancer, endometrial cancer, or glioblastoma. In one embodiment, the patient has a cancer that is refractory to one or more anti-cancer agents, e.g., gemcitabine or sunitinib.

In one embodiment the bispecific anti-IGF-1R and anti-ErbB3 antibody has an anti-IGF-1R module selected from the group consisting of SF, P4, M78, and M57. In another embodiment the bispecific anti-IGF-1R and anti-ErbB3 antibody has an anti-ErbB3 module selected from the group consisting of C8, P1, M1.3, M27, P6, and B69. In one embodiment, the bispecific anti-IGF-1R and anti-ErbB3 antibody is P4-G1-M1.3. In another embodiment, the bispecific anti-IGF-1R and anti-ErbB3 antibody is P4-G1-C8.

Also provided are methods of providing treatment of cancer in a human patient comprising co-administering to the patient an effective amount each of a bispecific anti-IGF-1R and anti-ErbB3 antibody and of one or more additional anti-cancer agents, wherein the anti-cancer agent is a PI3K pathway inhibitor, an mTOR inhibitor, a MEK inhibitor, a multikinase inhibitor, a B-Raf inhibitor, a taxane, irinotecan, nanoliposomal irinotecan, an anti-endocrine therapy, an antihormonal therapy, or an antimetabolite therapy. In some embodiments the anti-cancer agent is an mTOR inhibitor. Exemplary mTOR inhibitors are selected from the group comprising everolimus, temsirolimus, sirolimus, or ridaforolimus. In other embodiments the mTOR inhibitor is a pan-mTOR inhibitor selected from the group consisting of INK128, CC223, OSI207, AZD8055, AZD2014, and Palomid529. In some embodiments the anti-cancer agent is a phosphoinositide-3-kinase (PI3K) inhibitor or PI3K pathway inhibitor, e.g., perifosine (KRX-0401), SF1126, CAL101, BKM120, BKM120, XL147, or PX-866. In one embodiment, the PI3K inhibitor is XL147 or BKM120. In some embodiments, the anti-cancer agent is a MEK inhibitor, e.g., GSK1120212. In some embodiments, the anti-cancer agent is a multikinase inhibitor. In certain embodiments, the multikinase inhibitor is sorafenib. In some embodiments the anti-cancer agent is an antimetabolite therapy, e.g., gemcitabine, capecitabine, cytarabine, or 5-fluorouracil. In certain embodiments, the antimetabolite is gemcitabine. In other embodiments, the antimetabolite is a taxane such as docetaxel, cabazitaxel, nab-paclitaxel, or paclitaxel. In another embodiment, the antimetabolite is capecitabine or 5-fluorouracil. In some embodiments, the anti-cancer agent is irinotecan or nanoliposomal irinotecan. In another embodiment, the anti-cancer agent is a B-Raf inhibitor. In some embodiments, the anti-cancer agent is antihormonal therapy. In certain embodiments, then antihormonal therapy is tamoxifen, exemestane, letrozole, or fulvestrant.

In some embodiments, co-administration of the additional anti-cancer agent or agents has an additive or superadditive effect on suppressing tumor growth, as compared to administration of the bispecific anti-IGF-1R and anti-ErbB3 antibody alone or the one or more additional anti-cancer agents alone, wherein the effect on suppressing tumor growth is measured in a mouse xenograft model using BxPC-3, Caki-1, SK-ES-1, A549, NCI/ADR-RES, BT-474, DU145, or MCF7 cells.

Also provided are compositions for use in the treatment of a cancer, or for the manufacture of a medicament for the treatment of cancer, said composition comprising a bispecific anti-IGF-1R and anti-ErbB3 antibody to be administered to a patient requiring treatment of a cancer, the administration comprising administering to the patient a single loading dose of at least 10 mg/kg of the bispecific antibody followed by administration of one or more maintenance doses given at intervals. The intervals between doses are intervals of at least three days. In some embodiments, the intervals between doses are every fourteen days or every twenty-one days.

In some embodiments, the compositions comprise a loading dose that is greater than the maintenance dose. The loading dose may from about 12 mg/kg to about 20 mg/kg, from about 20 mg/kg to about 40 mg/kg, or from about 40 mg/kg to about 60 mg/kg. In some embodiments the loading dose is about 12 mg/kg, about 20 mg/kg, about 40 mg/kg, or about 60 mg/kg. In certain embodiments the maintenance dose is about 6 mg/kg, about 12 mg/kg, about 20 mg/kg, about 30 mg/kg, about 40 mg/kg, about 50 mg/kg or about 60 mg/kg. In one embodiment, the patient has a cancer that is refractory to one or more anti-cancer agents, e.g., gemcitabine, sunitinib, or sorafenib.

In some embodiments the patient has a pancreatic cancer, renal cell carcinoma, hepatocellular carcinoma, Ewing's sarcoma, non-small cell lung cancer, gastrointestinal neuroendocrine cancer, estrogen receptor-positive locally advanced or metastatic cancer, ovarian cancer, colorectal cancer, endometrial cancer, or glioblastoma.

In one embodiment the bispecific anti-IGF-1R and anti-ErbB3 antibody has an anti-IGF-1R module selected from the group consisting of SF, P4, M78, and M57. In another embodiment the bispecific anti-IGF-1R and anti-ErbB3 antibody has an anti-ErbB3 module selected from the group consisting of C8, P1, M1.3, M27, P6, and B69. In one embodiment, the bispecific anti-IGF-1R and anti-ErbB3 antibody is P4-G1-M1.3. In another embodiment, the bispecific anti-IGF-1R and anti-ErbB3 antibody is P4-G1-C8.

In some embodiments the compositions comprise an effective amount each of a bispecific anti-IGF-1R and anti-ErbB3 antibody and of one or more additional anti-cancer agents, wherein the anti-cancer agent is a PI3K pathway inhibitor, an mTOR inhibitor, a MEK inhibitor, a multikinase inhibitor, a B-Raf inhibitor, nanoliposomal irinotecan, or an antimetabolite. In some embodiments the anti-cancer agent is an mTOR inhibitor. In certain embodiments, the mTOR inhibitor is selected from the group comprising everolimus, temsirolimus, sirolimus, or ridaforolimus. In other embodiments the mTOR inhibitor is a pan-mTOR inhibitor chosen from the group consisting of INK128, CC223, OSI207, AZD8055, AZD2014, and Palomid529. In some embodiments the anti-cancer agent is a phosphoinositide-3-kinase (PI3K) inhibitor, e.g., perifosine (KRX-0401), SF1126, CAL101, BKM120, BKM120, XL147, or PX-866. In one embodiment, the PI3K inhibitor is XL147. In some embodiments, the anti-cancer agent is a MEK inhibitor. Exemplary MEK inhibitors are selected from the group consisting of GSK1120212, BAY 86-9766, or AZD6244. In some embodiments, the anti-cancer agent is a multikinase inhibitor. In certain embodiments, the multikinase inhibitor is sorafenib or sunitinub. In some embodiments the anti-cancer agent is an antimetabolite, e.g., gemcitabine, docetaxel, paclitaxel, capecitabine, cytarabine, or 5-fluorouracil. In one embodiment, the anti-cancer agent is nanoliposomal irinotecan. In another embodiment, the anti-cancer agent is a B-Raf inhibitor.

In some embodiments the composition comprises a bispecific anti-IGF-1R and anti-ErbB3 antibody and one or more additional anti-cancer agents, wherein co-administration of the anti-cancer agent or agents has an additive or superadditive effect on suppressing tumor growth, as compared to administration of the bispecific anti-IGF-1R and anti-ErbB3 antibody alone or the one or more additional anti-cancer agents alone, wherein the effect on suppressing tumor growth is measured in a mouse xenograft model using BxPC-3, Caki-1, SK-ES-1, A549, NCI/ADR-RES, BT-474, DU145, or MCF7 cells.

Also provided are kits comprising a therapeutically effective amount of a bispecific anti-IGF-1R and anti-ErbB3 antibody and a pharmaceutically-acceptable carrier. The kits further comprise instructions to a practitioner, wherein the instructions comprise dosages and administration schedules for the bispecific anti-IGF-1R and anti-ErbB3 antibody. In one embodiment, the kit includes multiple packages each containing a single dose amount of the antibody. In another embodiment, the kit provides infusion devices for administration of the bispecific anti-IGF-1R and anti-ErbB3 antibody. In another embodiment, the kit further comprises an effective amount of at least one additional anti-cancer agent.

### BRIEF DESCRIPTION OF THE DRAWINGS

FIG. 1 is a graph demonstrating the inhibition of growth of Caki-1 renal cell carcinoma cancer cells in vivo by P4-G1-M1.3 (500 μg, 300 μg, or 100 μg) the mTOR inhibitor (mTORi) everolimus (30mpk or 3mpk), or the combination of everolimus (3mpk) and P4-G1-M1.3 (500 μg). The y-axis represents mean tumor volume in mm³ and the x-axis represents time in days.

FIGS. 2A-2J are graphs demonstrating the level of IGF-1R and insulin receptor (FIG. 2A), EGFR and ErbB3 (FIG. 2B), ErbB2 (FIG. 2C), phospho-AKT (pAKT, Ser473 and Thr308) (FIG. 2D), phospho-Fox01 (Thr24)/Fox03a (Thr32) and phospho-PDK1 (pPDK1) (FIG. 2E), phospho-mTOR (p-mTOR) Ser2448 and Ser2481 (FIG. 2F), pS6 (Ser235/236 and Ser240/244)(FIG. 2G), phospho-ERK (p-ERK) and survivin (FIG. 2H), phospho-PRAS40 (Ser183 and Thr246)(FIG. 2I), phospho-4E-BP1 p4E-BP1) (Thr37/46 and Ser65) (FIG. 2J), in end of study BxPC-3 tumors of mice in which one of PBS, P4-G1-M1.3, gemcitabine, or P4-G1-M1.3+gemcitabine (combined individual doses) was administered.

FIGS. 3A-3D are graphs demonstrating the level of pAkt Ser473 (FIG. 3A, FIG. 3B) and pERK (FIG. 3C, FIG. 3D) in BxPC-3 cells (FIG. 3A, FIG. 3C) wild-type for KRAS or KP4 cells (FIG. 3B, FIG. 3D) mutant for KRAS. Cells were treated with 500 nM P4-G1-M1.3, 250 nM GSK1120212 or the combination for 24 hours in 10% serum and ELISA assays were performed. The data was normalized to 10% serum without treatment.

FIG. 4 is a graph that demonstrates the inhibition of growth of DU145 prostate cancer cells in vivo by P4-G1-M1.3 alone (30mpk, q3d), docetaxel alone (10mpk q7d), or the combination of docetaxel and P4-G1-M1.3. The y-axis represents mean tumor volume in mm³ and the x-axis represents time in days.

FIGS. 5A-5D are graphs that demonstrate the level of ErbB3 (FIG. 5A), pErbB3 (FIG. 5B), pAkt Ser473 (FIG. 5C) and pERK1/2 (FIG. 5D) in HepG2 hepatocellular carcinoma cells. Cells were treated with 500 nM P4-G1-M1.3, 504 sorafenib or the combination for either 2 hours or 6 hours and quantitative western blotting was performed.

FIG. 6 is a graph that represents the in vivo effects of P4-G1-M1.3 alone, docetaxel alone, or the combination of P4-G1-M1.3 and docetaxel on total IGF-1R in DU145 xenografts. Statistical significance across groups was determined using the student's T-test (*,p<0.05 vs control.; #,p<0.05 vs Docetaxel; a,p<0.05 vs P4-G1-M1.3).

FIG. 7 is a graph that represents the in vivo effects of P4-G1-M1.3 alone, docetaxel alone, or the combination of P4-G1-M1.3 and docetaxel on total ErbB3 in DU145 xenografts. Statistical significance across groups was determined using the student's T-test (*, p<0.05 vs control; #,p<0.05 vs Docetaxel; a,p<0.05 vs P4-G1-M1.3).

### DETAILED DESCRIPTION

### Methods and Compositions

Methods of monotherapy, combination therapy, monotherapeutic compositions, and combination compositions for treating cancer in a patient are provided. In these methods, the cancer patient is treated with both a bispecific anti-IGF-1R and anti-ErbB3 antibody and one or more additional anti-cancer agents selected, e.g., from an mTOR inhibitor, a MEK inhibitor, a multikinase inhibitor, a B-Raf inhibitor, nanoliposomal irinotecan, a PI3K inhibitor, and an antimetabolite.

The term “combinatorially enhanced” means that combination therapy with an effective amount of a first agent and an effective amount of a second agent provides a benefit that is greater than the benefit obtained in two matched comparisons: one in which the same effective amount of the first agent alone is separately administered as monotherapy to separate matched subjects and the other in which the same effective amount of the second agent alone is separately administered as monotherapy to separate matched subjects. Such a greater benefit may be seen in patients treated with the combination therapy as an improved therapeutic outcome compared to either of the monotherapy comparators, or as a therapeutic outcome that is equal to or better than that of either of the monotherapy comparators and is associated in the combination therapy with a reduction of adverse events as compared to the adverse events seen with either of the monotherapy comparators. An exemplary combinatorially enhanced outcome is one in which the greater benefit is a statistically significantly greater benefit with a p value of 0.05 or better, and each combinatorially enhanced outcome recited in the examples optionally corresponds to a statistically significantly greater benefit with a p value less than or equal to 0.05.

The terms “combination therapy,” “co-administration,” “co-administered” or “concurrent administration” (or minor variations of these terms) include simultaneous administration of at least two therapeutic agents to a patient or their sequential administration within a time period during which the first administered therapeutic agent is still present in the patient when the second administered therapeutic agent is administered.

The term “monotherapy” refers to administering a single drug to treat a disease or disorder in the absence of co-administration of any other therapeutic agent that is being administered to treat the same disease or disorder.

“Additional anti-cancer agent” is used herein to indicate any drug that is useful for the treatment of a malignant pancreatic tumor other than a drug that inhibits heregulin binding to ErbB2/ErbB3 heterodimer.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., taken at a single administration, e.g., orally (e.g., as one, two, three or more pills, capsules, etc.) or injected (e.g., as a bolus). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

“Effective amount” refers to an amount (administered in one or more doses) of an antibody, protein or additional therapeutic agent, which amount is sufficient to provide effective treatment.

“ErbB3” and “HER3” refer to ErbB3 protein, as described in U.S. Pat. No. 5,480,968. The human ErbB3 protein sequence is shown in SEQ ID NO:4 of U.S. Pat. No. 5,480,968, wherein the first 19 amino acids (aas) correspond to the leader sequence that is cleaved from the mature protein. ErbB3 is a member of the ErbB family of receptors, other members of which include ErbB1 (EGFR), ErbB2 (HER2/Neu) and ErbB4. While ErbB3 itself lacks tyrosine kinase activity, it can be phosphorylated upon dimerization with another ErbB family receptor, e.g., ErbB1, ErbB2 and ErbB4, which are receptor tyrosine kinases. Ligands for the ErbB family include heregulin (HRG), betacellulin (BTC), epidermal growth factor (EGF), heparin-binding epidermal growth factor (HB-EGF), transforming growth factor alpha (TGF-a), amphiregulin (AR), epigen (EPG) and epiregulin (EPR). The aa sequence of human ErbB3 is provided at Genbank Accession No. NP_001973.2 (receptor tyrosine-protein kinase erbB-3 isoform 1 precursor) and is assigned Gene ID: 2065.

“IGF-1R” or “IGF1R” refers to the receptor for insulin-like growth factor 1 (IGF-1, formerly known as somatomedin C). IGF-1R also binds to, and is activated by, insulin-like growth factor 2 (IGF-2). IGF1-R is a receptor tyrosine kinase, which upon activation by IGF-1 or IGF-2 is auto-phosphorylated. The aa sequence of the human IGF-1R precursor is provided at Genbank Accession No. NP_000866 and is assigned Gene ID: 3480.

“Module” refers to a structurally and/or functionally distinct part of a PBA, such a binding site (e.g., an scFv domain or a Fab domain) and the Ig constant domain. Modules provided herein can be rearranged (by recombining sequences encoding them, either by recombining nucleic acids or by complete or fractional de novo synthesis of new polynucleotides) in numerous combinations with other modules to produce a wide variety of PB As, e.g., as disclosed herein. For example, an “SF” module refers to the binding site “SF,” i.e., comprising at least the CDRs of the SF VH and SF VL domains. A “C8” module refers to the binding site “C8.”

“PBA” refers to a polyvalent bispecific antibody, an artificial hybrid protein comprising at least two different binding moieties or domains and thus at least two different binding sites (e.g., two different antibody binding sites), wherein one or more of the pluralities of the binding sites are covalently linked, e.g., via peptide bonds, to each other. A preferred PBA described herein is an anti-IGF-1R+anti-ErbB3 PBA, which is a polyvalent bispecific antibody that comprises one or more first binding sites binding specifically to an IGF-1R protein, e.g., a human IGF-1R protein, and one or more second binding sites binding specifically to an ErbB3 protein, e.g., a human ErbB3 protein. An anti-IGF-1R+anti-ErbB3 PBA is so named regardless of the relative orientations of the anti-IGF-1R and anti-ErbB3 binding sites in the molecule, whereas when the PBA name comprises two antigens separated by a slash (/) the antigen to the left of the slash is amino terminal to the antigen to the right of the slash. A PBA may be a bivalent binding protein, a trivalent binding protein, a tetravalent binding protein or a binding protein with more than 4 binding sites. An exemplary PBA is a tetravalent bispecific antibody, i.e., an antibody that has 4 binding sites, but binds to only two different antigens or epitopes. Exemplary bispecific antibodies are tetravalent “anti-IGF-1R/anti-ErbB3” PBAs and “anti-ErbB3/anti-IGF-1R” PBAs. Typically the N-terminal binding sites of a tetravalent PBA are Fabs and the C-terminal binding sites are scFvs. IGF-1R+ErbB3 PBAs comprising IgG1 constant regions each comprise two joined essentially identical subunits, each subunit comprising a heavy and a light chain that are disulfide bonded to each other, e.g., M7-G1-M78 (SEQ ID NO:146 and SEQ ID NO:147), P4-G1-M1.3 (SEQ ID NO:148 and SEQ ID NO:149), and P4-G1-C8 (SEQ ID NO:150 and SEQ ID NO:151), are exemplary embodiments of such IgG1-(scFv)₂proteins. When the immunoglobulin constant regions are those of IgG2, the protein is referred to as an IgG2-(scFv)₂. Other exemplary IGF-1R+ErbB3 PBAs comprising IgG1 constant regions include, e.g., SF-G1-P1,SF-G1-M1.3, SF-G1-M27, SF-G1-P6, SF-G1-B69, P4-G1-C8, P4-G1-P1, P4-G1-M1.3, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, P1-G1-M76 (set forth in the Appendix enclosed herewith, and incorporated by reference herein).

Combination Therapies with Additional Anti-Cancer Agents

As herein provided, BPAs (e.g., P4-G1-M1.3) are co-administered with one or more additional anti-cancer agents (e.g., an mTOR inhibitor, a MEK inhibitor, a multikinase inhibitor, a B-Raf inhibitor, an anti-endocrine therapy, antihormonal therapy, irinotecan or nanoliposomal irinotecan, a PI3K inhibitor, or an antimetabolite), to provide effective treatment to human patients having a cancer (e.g., pancreatic, ovarian, lung, colon, head and neck, and esophageal cancers).

Additional anti-cancer agents suitable for combination with anti-IGF-1R+anti-ErbB3 antibodies may include but are not limited to pyrimidine antimetabolites (e.g., the nucleoside metabolic inhibitor gemcitabine, cytarabine, or the pyrimidine analog 5-fluorouracil), mTOR inhibitors (e.g., everolimus, temsirolimus, sirolimus, or ridaforolimus), pan-mTOR inhibitors (e.g., INK128, CC223, OSI207, AZD8055, AZD2014, or Palomid529), phosphoinositide-3-kinase (PI3K) inhibitors (e.g., perifosine (KRX-0401), SF1126, CAL101, BKM120, BKM120, XL147, and PX-866), MEK inhibitors (e.g., GSK1120212, BAY 86-9766 or AZD624), taxanes (e.g., paclitaxel, nab-paclitaxel, cabazitaxel, and docetaxel), and nanoliposomal irinotecan (e.g., MM-398).

In certain combination therapy methods, one or more of the following therapeutic agents is co-administered to the patient with an anti-IGF-1R+anti-ErbB3 antibody.

Gemcitabine (Gemzar®) is indicated as first line therapy for pancreatic adenocarcinoma and is also used in various combinations to treat ovarian, breast and non-small-cell lung cancers. Gemcitabine HCl is 2′-deoxy-2′,2′-difluorocytidine monohydrochloride (-isomer) (MW=299.66) and is administered parenterally, typically by i.v. infusion.

Temsirolimus (Torisel®) is an mTOR inhibitor that is administered parenterally, typically by i.v. infusion and is used to treat advanced renal cell carcinoma.

Everolimus (Afinitor®), a 40-O-(2-hydroxyethyl) derivative of sirolimus, is an mTOR inhibitor that is administered orally and is used to treat progressive neuroendocrine tumors of pancreatic origin (PNET) in patients with unresectable, locally advanced or metastatic disease.

5-Fluorouracil (5-FU Adrucil®, Carac®, Efudix®, Efudex® and Fluoroplex®) is a pyrimidine analog that works through irreversible inhibition of thymidylate synthase.

Capecitabine (Xeloda®) is an orally administered systemic prodrug of 5′-deoxy-5-fluorouridine (5′-DFUR) which is converted to 5-fluorouracil.

Docetaxel (Taxotere®) is an anti-mitotic chemotherapy used for the treatment of breast, advanced non-small cell lung, metastatic androgen-independent prostate, advanced gastric and locally advanced head and neck cancers.

Paclitaxel (Taxol®) is an anti-mitotic chemotherapy used for the treatment of lung, ovarian, breast and head and neck cancers.

Sorafenib (Nexavar®) is a small molecule inhibitor of multiple tyrosine kinases (including VEGFR and PDGFR) and Raf kinases (an exemplary “multikinase inhibitor”) used for treatment of advanced renal cell carcinoma (RCC) and advanced primary liver cnacer (hepatocellular carcinoma, HCC).

Trametinib (GSK-1120212) is a small molecule inhibitor of the MEK protein currently in clinical trials for the treatment of several cancers including pancreatic, melanoma, breast and non-small cell lung.

Vemurafenib (Zelboraf®) is a small molecule inhibitor of B-Raf in patients whose cancer cells harbor a V600E B-Raf mutation. Vemurafenib is currently approved for treatment of late-stage, unresectable, and metastatic melanoma.

Nanoliposomal irinotecan (e.g., MM-398) is a stable nanoliposomal formulation of irinotecan. MM-398 is described, e.g., in U.S. Pat. No. 8,147,867. MM-398 may be administered, for example, on day 1 of the cycle at a dose of 120 mg/m2, except if the patient is homozygous for allele UGT1A1*, wherein nanoliposomal irinotecan is administered on day 1 of cycle 1 at a dose of 80 mg/m².The required amount of MM-398 may be diluted, e.g., in 500 mL of 5% dextrose injection USP and infused over a 90 minute period.

### Outcomes

As shown in the Examples herein, co-administration of an anti-IGF-1R+anti-ErbB3 antibody with one or more additional therapeutic agents (e.g., everolimus, temsirolimus, sirolimus, XL147, gemcitabine, 5-fluorouracil, cytarabine) provides improved efficacy compared to treatment with the antibody alone or with the one or more additional therapeutic agents in the absence of antibody therapy. Preferably, a combination of an anti-IGF-1R+anti-ErbB3 antibody with one or more additional therapeutic agents exhibits therapeutic synergy.

“Therapeutic synergy” refers to a phenomenon where treatment of patients with a combination of therapeutic agents manifests a therapeutically superior outcome to the outcome achieved by each individual constituent of the combination used at its optimum dose (T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

Thus, in combination, the components of such combinations have an additive or superadditive effect on suppressing tumor growth, as compared to monotherapy with the PBA or treatment with the chemotherapeutic(s) in the absence of antibody therapy. By “additive” is meant a result that is greater in extent (e.g., in the degree of reduction of tumor mitotic index or of tumor growth or in the degree of tumor shrinkage or the frequency and/or duration of symptom-free or symptom-reduced periods) than the best separate result achieved by monotherapy with each individual component, while “superadditive” is used to indicate a result that exceeds in extent the sum of such separate results. In one embodiment, the additive effect is measured as slowing or stopping of tumor growth. The additive effect can also be measured as, e.g., reduction in size of a pancreatic tumor, reduction of tumor mitotic index, reduction in number of metastatic lesions over time, increase in overall response rate, or increase in median or overall survival. One non-limiting example of a measure by which effectiveness of a therapeutic treatment can be quantified is by calculating the log10 cell kill, which is determined according to the following equation:

log10 cell kill=T C(days)/3.32×Td

in which T C represents the delay in growth of the cells, which is the average time, in days, for the tumors of the treated group (T) and the tumors of the control group (C) to have reached a predetermined value (1 g, or 10 mL, for example), and Td represents the time, in days necessary for the volume of the tumor to double in the control animals. When applying this measure, a product is considered to be active if log10 cell kill is greater than or equal to 0.7 and a product is considered to be very active if log10 cell kill is greater than 2.8. Using this measure, a combination, used at its own maximum tolerated dose, in which each of the constituents is present at a dose generally less than or equal to its maximum tolerated dose, exhibits therapeutic synergy when the log10 cell kill is greater than the value of the log10 cell kill of the best constituent when it is administered alone. In an exemplary case, the log10 cell kill of the combination exceeds the value of the log10 cell kill of the best constituent of the combination by at least 0.1 log cell kill, at least 0.5 log cell kill, or at least 1.0 log cell kill.

### Kits and Unit Dosage Forms

Further provided are kits that include a pharmaceutical composition containing a bispecific anti-IGF-1R and anti-ErbB3 antibody, including a pharmaceutically-acceptable carrier, in a therapeutically effective amount adapted for use in the preceding methods. The kits include instructions to allow a practitioner (e.g., a physician, nurse, or patient) to administer the composition contained therein to treat an ErbB2 expressing cancer.

Preferably, the kits include multiple packages of the single-dose pharmaceutical composition(s) containing an effective amount of a bispecific anti-IGF-1R and anti-ErbB3 antibody for a single administration in accordance with the methods provided above. Optionally, instruments or devices necessary for administering the pharmaceutical composition(s) may be included in the kits. For instance, a kit may provide one or more pre-filled syringes containing an amount of a bispecific anti-IGF-1R and anti-ErbB3 antibody that is about 100 times the dose in mg/kg indicated for administration in the above methods.

Furthermore, the kits may also include additional components such as instructions or administration schedules for a patient suffering from a disease or condition (e.g., a cancer, autoimmune disease, or cardiovascular disease) to use the pharmaceutical composition(s) containing a bispecific anti-IGF-1R and anti-ErbB3 antibody, or any binding, diagnostic, and/or therapeutic agent conjugated thereto.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The following Examples should not be construed as limiting the scope of this disclosure.

### Materials and Methods

Throughout the Examples, the following materials and methods are used unless otherwise stated. In general, the practice of the techniques of the present disclosure employs conventional methods of drug administration, and, unless otherwise indicated, conventional techniques of chemistry, molecular biology, recombinant DNA technology, immunology (especially, e.g., antibody technology), pharmacology, pharmacy, and standard techniques in polypeptide preparation.

### Cell Lines

All the human cell lines for use in the experiments described below may be obtained, as indicated. With one exception these are from American Type Culture Collection (ATCC, Manassas, VA) or the US National Cancer Institute (NCI) e.g., from the Division of Cancer Treatment and Diagnostics (DCTD).
A549—ATCC® cat. No. CCL-185™
ADRr-NCI (redesignated NCI/ADR-RES)
BT-474—ATCC® cat. No. HTB-20™
BxPC-3—ATCC® cat. No. CRL1687™
Caki-1—ATCC® cat. No. HTB-46™
DU145—ATCC® cat. No. HTB-81™
SK-ES-1—ATCC® cat. No. HTB-86™
MCF7—ATCC® cat. No. HTB-22™
KP4—RIKEN cat. No. RCB1005
HepG2—ATCC® cat. No. HB8065™

### Xenograft Studies

For each of the xenograft studies below, cells are resuspended 1:1 with PBS:Growth factor-reduced Matrigel® and injected subcutaneously into Nu/Nu mice. Tumors are allowed to develop for 8 days. Antibodies are injected intraperitoneally every 3 days (q3d) at the indicated doses/mouse. Tumor lengths and widths are measured twice a week manually by caliper, and tumor volume calculated using the following formula: π/6(L×W²). Each arm of a study contains 10 animals. All studies are carried out using methods approved by an internal IACUC panel.

### Pharmacodynamic Profiling in Xenografts

BxPC-3 mouse xenograft models are established using 5×10⁶ BxPC-3 cells that are resuspended 1:1 with PBS:Growth factor-reduced Matrigel® and injected subcutaneously into Nu/Nu mice. Tumors are allowed to develop for 8 days. Antibodies are injected intraperitoneally every 3 days (q3d) for 2 rounds of dosing.

For the BxPC-3 PD study, 4 treatment groups are established, each containing 4 mice. These included control, P4-G1-M1.3 (q3d, 600 μg), gemcitabine (q3d, 150 mg/kg) and P4-G1-M1.3+gemcitabine (combined individual doses). Tumors are excised on either day 19 or day 28, resulting in a total of 8 groups.

### Preparation of Tumor Cell Lysates

Tumors are initially weighed and pulverized in a CryoPrep® tissue pulverizer (Covaris). Tissue Extraction Reagent 1 (TER1, Life Technologies™) containing protease and phosphatase inhibitors was added to the tumor at a ratio of lml TER1 per 100 mg of tissue. Samples are incubated on ice for 30 minutes to solubilize tissue and put through a QIAshredder™ column (Qiagen) according to the manufacturer's protocol. A BCA assay (Pierce) is performed to determine protein concentration according to the manufacturer's protocol.

### Western Blotting

Buffer containing β-Mercaptoethanol (β-ME) is added and lysates are boiled for 5 minutes at 95° C. Approximately 40 μg of protein and two ladders (Invitrogen) are run on each well of an 18-well gel (BioRad). Gels are run at 150 volts constant for approximately 90 minutes and transferred to nitrocellulose membranes using the 8 minute transfer program of the iBlot® (Invitrogen) transfer system. Membranes are blocked in Odyssey® Blocking Buffer (Licor® Biosciences) for 1 hour at room temperature, and then incubated with primary antibodies overnight at 4° C. in 5% BSA in TBS-T. All antibodies are purchased from Cell Signaling and used at the recommended dilution. The following day membranes are washed 3×5 minutes each with TBS-T and then incubated with anti-Rabbit IgG—DyLight® 800 (Cell Signaling) or anti-Rabbit IRDye® 800 (Licor® Biosciences) at 1:10,000-15,000 in 5% milk in TBS-T for 1 hour at room temperature. Membranes were then washed 3×5 minutes each with TBS-T and scanned using the Licor® Odyssey® system (Licor® Biosciences). Intensities are quantified using Image Studio 2.0 and normalized to β-Actin levels.

### Example 1

Patients with renal cell carcinoma are treated by administration of monotherapy with either an effective amount of mTOR inhibitor everolimus (Afinitor®) or an effective amount of P4-G1-M1.3, or with combination therapy comprising or consisting of administration of both the effective amount of everolimus and the effective amount of P4-G1-M1.3.

P4-G1-M1.3 is formulated in 20 mM histidine, 100 mM arginine-HCl, 3% sucrose, at pH 5.5 supplemented with 0.002-0.02% of Tween® 80 at concentration range 5-15 mg/mL. P4-G1-M1.3 is administered to patients at 6 mg/kg, 12 mg/kg, 20 mg/kg, 30 mg/kg, 40 mg/kg, 50 mg/kg or 60 mg/kg q7d, ql4d, q21d, or q28d with a loading dose of 12 mg/kg, 20 mg/kg, 40 mg/kg, 40 mg/kg or 60 mg/kg Everolimus is administered to patients at 2.5 mg, 5 mg, or 10 mg orally once a day or once every other day.

The combination therapy will provide a combinatorially enhanced outcome.

### Example 2

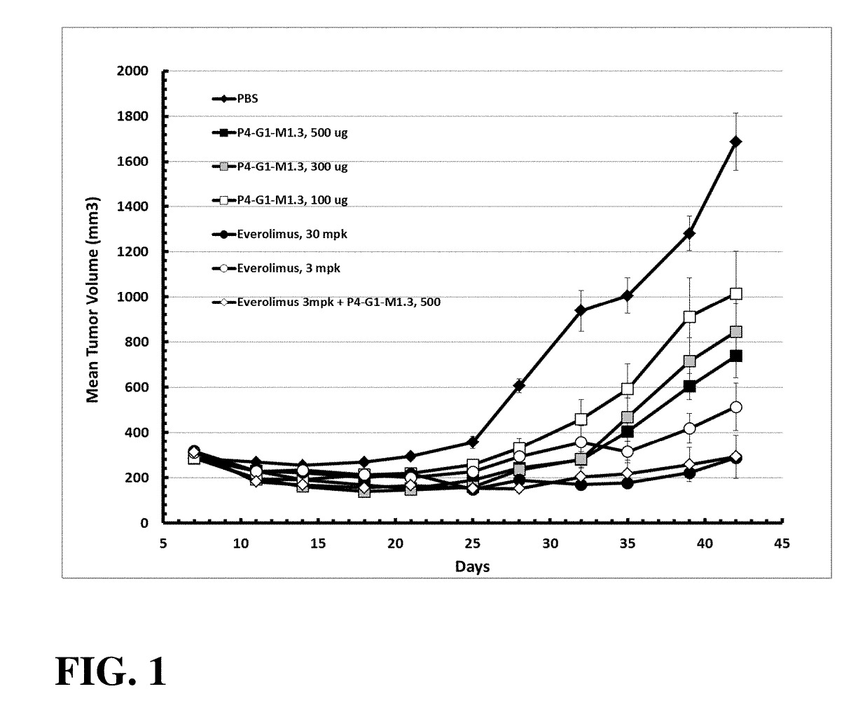

The advantages of combination therapy per Example 1 are demonstrated in a preclinical model. 8×10⁶ Caki-1 human renal carcinoma cells were prepared and used essentially as described in the methods above and mice were treated with P4-G1-M1.3 at 500, 300, or 100 μg, or everolimus at 30 mpk or 3 mpk, or 3mpk everolimus+500 μg P4-G1-M1.3. As shown in FIG. 1, P4-G1-M1.3 suppresses tumor growth of Caki-1 renal cell carcinoma cancer cells in vivo and potentiates responses to everolimus.

### Example 3

Patients with gastrointestinal neuroendocrine tumors are treated by administration of either monotherapy with an effective amount of everolimus or an effective amount of P4-G1-M1.3, or with combination therapy comprising or consisting of administration of both the effective amount of everolimus and the effective amount of P4-G1-M1.3. P4-G1-M1.3 and everolimus are prepared and dosed as described in Example 1. The combination therapy will provide a combinatorially enhanced outcome.

### Example 4

The advantages of combination therapy per Example 3 are demonstrated in a preclinical model carried out using the methods of Example 2 adapted for the substitution of human pancreatic adenocarcinoma BXPC-3 cells for the Caki-1 cells of Example 2. The results will demonstrate that P4-G1-M1.3 suppresses tumor growth of BXPC-3 cells in vivo and potentiates responses to everolimus.

### Example 5

Patients with non-small cell lung cancer (NSCLC) are treated by administration of either monotherapy with an effective amount of everolimus or an effective amount of P4-G1-M1.3, or with combination therapy comprising or consisting of administration of both the effective amount of everolimus and the effective amount of P4-G1-M1.3. P4-G1-M1.3 and everolimus are prepared and dosed as described in Example 1. The combination therapy will provide a combinatorially enhanced outcome.

### Example 6

The advantages of combination therapy per Example 5are demonstrated in a preclinical model carried out using the methods of Example 2 adapted for the substitution of human NSCLC A549 cells for the Caki-1 cells of Example 2. The results will demonstrate that P4-G1-M1.3 suppresses tumor growth of A549 cells in vivo and potentiates responses to everolimus.

### Example 7

Patients with gastrointestinal neuroendocrine tumors are treated by administration of either monotherapy with an effective amount of mTOR inhibitor temsirolimus (Torisel®) or an effective amount of P4-G1-M1.3, or with combination therapy comprising or consisting of administration of both the effective amount of temsirolimus and the effective amount of P4-G1-M1.3. P4-G1-M1.3 is prepared and dosed as described in Example 1. Temsirolimus is dosed at 2.5 mg, 7.5 mg, 15 mg, or 25 mg (25 mg is the manufacturer's recommended dose) infused over a 30-60 minute period once a week. The combination therapy will provide a combinatorially enhanced outcome.

### Example 8

The advantages of combination therapy per Example 7 are demonstrated in a preclinical model carried out using the methods of Example 2 adapted for the substitution of human pancreatic adenocarcinoma BXPC-3 cells for the Caki-1 cells of Example 2. The results will show that P4-G1-M1.3 suppresses tumor growth of BXPC-3 cells in vivo and potentiates the response to everolimus.

### Example 9

The advantages of combination therapy per Example 1 are demonstrated in animal models where the tumor type Ewing's sarcoma family of tumors, or renal cell carcinoma (second line in patients refractory to sunitinib).

### Example 10

The advantages of combination therapy per Example 9 are demonstrated in a preclinical model carried out using the methods of Example 2 adapted for the substitution of SK-ES-1 human Ewing sarcoma cells for the Caki-1 cells of Example 2. The results will show that P4-G1-M1.3 suppresses tumor growth of SK-ES-1 cells in vivo and potentiates responses to everolimus.

### Example 11

Patients with gastrointestinal neuroendocrine tumors are treated by administration of either monotherapy with an effective amount of mTOR inhibitor sirolimus (Rapamune®) or an effective amount of P4-G1-M1.3, or with combination therapy comprising or consisting of administration of both the effective amount of sirolimus and the effective amount of P4-G1-M1.3. P4-G1-M1.3 is prepared and dosed as described in Example 1. Patients are dosed with sirolimus at 0.2 mg, 0.5 mg, 2 mg, 5 mg, 10 mg, 15 mg, or 20 mg orally once a day with a loading dose of 0.6 mg, 1.5 mg, 6 mg, 15 mg, or 30 mg (3× the maintenance dose. The combination therapy will provide a combinatorially enhanced outcome.

### Example 12

The advantages of combination therapy per Example 11 are demonstrated in a preclinical model carried out using the methods of Example 2. The results will show that P4-G1-M1.3 suppresses tumor growth of Caki-1 cells in vivo and potentiates responses to sirolimus.

### Example 13

Patients with estrogen receptor positive or progesterone receptor positive or triple negative breast cancers that are locally advanced or metastatic, or with any of the tumor types listed in Examples 5-10, are treated with a combination of an effective amount of i) any of the mTOR inhibitors of the preceding examples (at doses as described therein), or with a pan-mTOR inhibitor (INK128, CC223, OSI207, AZD8055, AZD2014, or Palomid529) and ii) an effective amount of P4-G1-M1.3. Patients are dosed with P4-G1-M1.3 as described in Example 1. The dose of pan-mTOR inhibitor is the dose used in phase I or, preferably phase II or III clinical trials. The combination therapy will provide a combinatorially enhanced outcome.

### Example 14

Postmenopausal women with estrogen receptor-positive locally advanced or metastatic breast cancer are treated with a combination of an effective amount of PI3K inhibitor (e.g., XL147 or BKM120) and an effective amount of P4-G1-M1.3. Patients are dosed with P4-G1-M1.3 as described in Example 1. XL147 is dosed at 25, 50, 100, or 200 mg orally once a day for 21 consecutive days. Alternately BKM120 is dosed at 12.5, 25, 50, 100, or 20 mg orally once a day for 28 consecutive days. The combination therapy will provide a combinatorially enhanced outcome.

### Example 15

The advantages of combination therapy per Examples 13 and 14 are demonstrated in preclinical models carried out using the methods of Example 2 adapted for the use of MCF7 or BT474-M3 human ER/PR positive breast cancer cells. The results will demonstrate that P4-G1-M1.3 suppresses tumor growth of MCF7 cells and BT474-M3 cells in vitro and in vivo and potentiates responses to the PI3K inhibitors and the mTOR inhibitors of the preceding Examples.

### Example 16

Women with estrogen or progesterone receptor positive locally advanced or metastatic breast cancer are treated with a combination therapy comprising or consisting of administration of an effective amount of an antihormonal therapy (such as tamoxifen, exemestane, letrozole or fulvestrant) and administration of an effective amount of P4-G1-M1.3. P4-G1-M1.3 is formulated and administered as described above. Antihormonal therapy is administered in accordance with manufacturer's directions. The combination therapy will provide a combinatorially enhanced outcome.

### Example 17

Women with estrogen or progesterone receptor positive locally advanced or metastatic breast cancer are treated with a combination of an effective amount of PI3K/mTOR dual inhibitor NVP-BEZ235 and an effective amount of P4-G1-M1.3. Patients are dosed with P4-G1-M1.3 as described in Example 1. NVP-BEZ235 is given orally twice daily at doses of 400 mg, 600 mg, or 800 mg. The combination therapy will provide a combinatorially enhanced outcome.

The preclinical models to demonstrate the working of this Example is adapted from Examples 2, 4, 6, 8, 10, 12, and 15 using the ZR-75-1 human breast cancer cells and Caki-1 cells.

### Example 18

The combination therapies of Examples 1, 11, 13, 14 and 17 are expanded to include patients in which the tumor type can be pancreatic cancer, Ewing's sarcoma family of tumors, NSCLC, renal cell carcinoma (second line in renal carcinoma patients refractory to sunitinib (Sutent®)), or estrogen or progesterone receptor positive locally advanced or metastatic breast cancer.

Preclinical models to demonstrate these combination effects are carried out using MCF7, BT474-M3, BxPC-3, SK-ES-1, A549, CAM-1, and ZR-75-1 cells in vitro and in vivo.

### Example 19

Patients with pancreatic carcinoma are treated with a combination of an effective amount of gemcitabine, cytarabine, capecitabine, or 5-fluorouracil (5-FU) and an effective amount of P4-G1-M1.3. Patients are dosed with P4-G1-M1.3 as described in Example 1. Patients are dosed with the manufacturer's recommended dose of gemcitabine, capecitabine or 5-FU. The combination therapy will provide a combinatorially enhanced outcome.

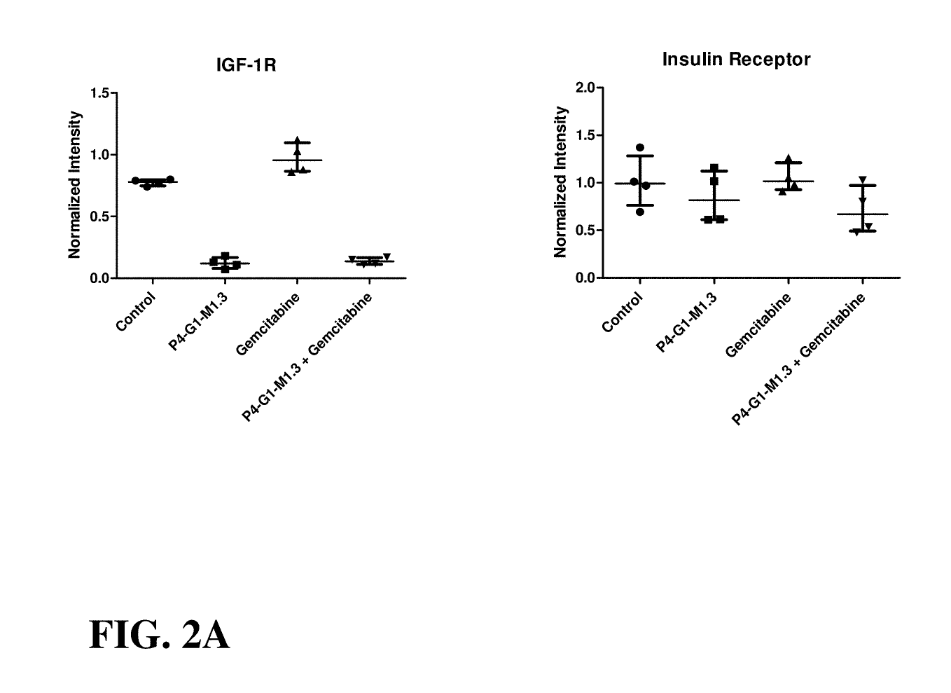

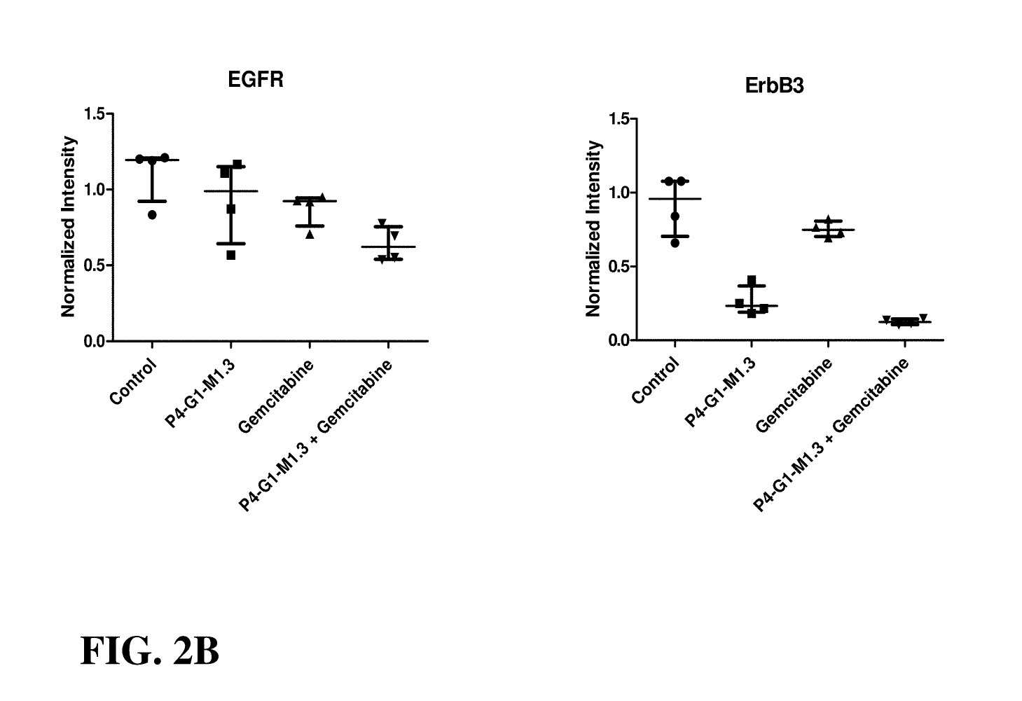

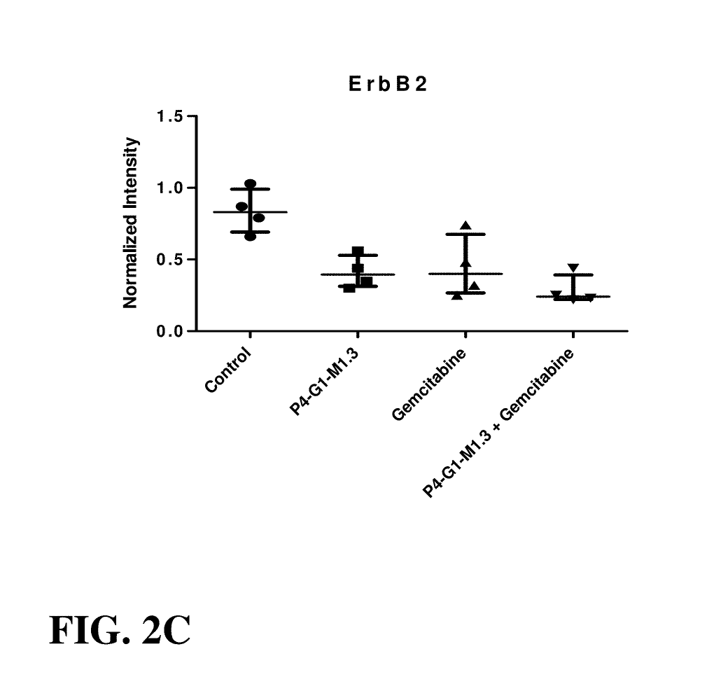

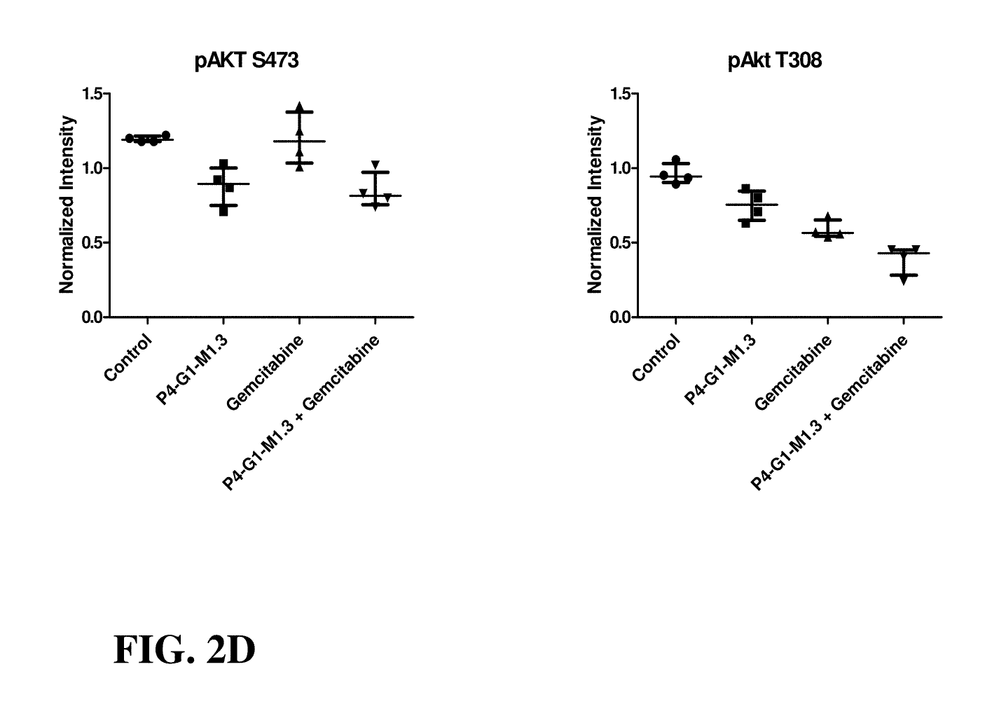

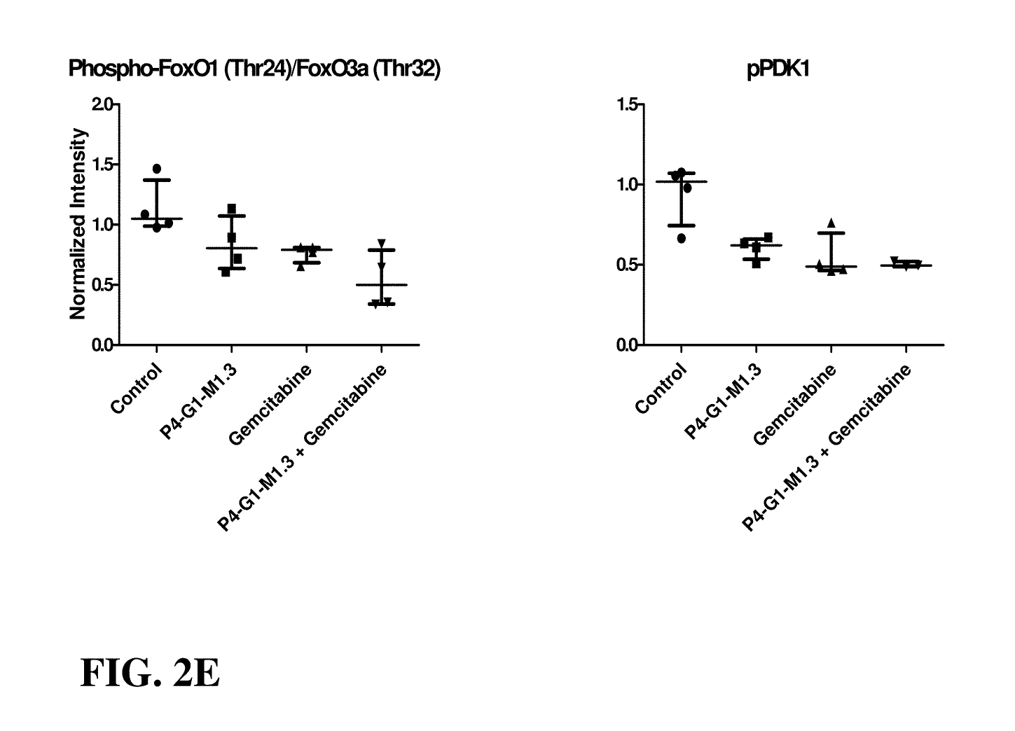

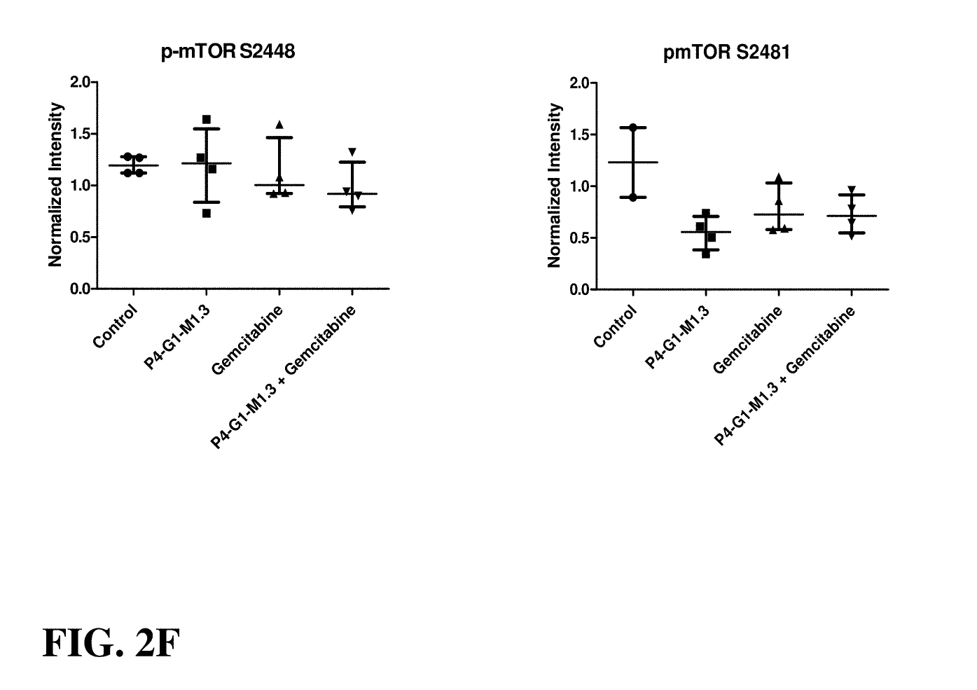

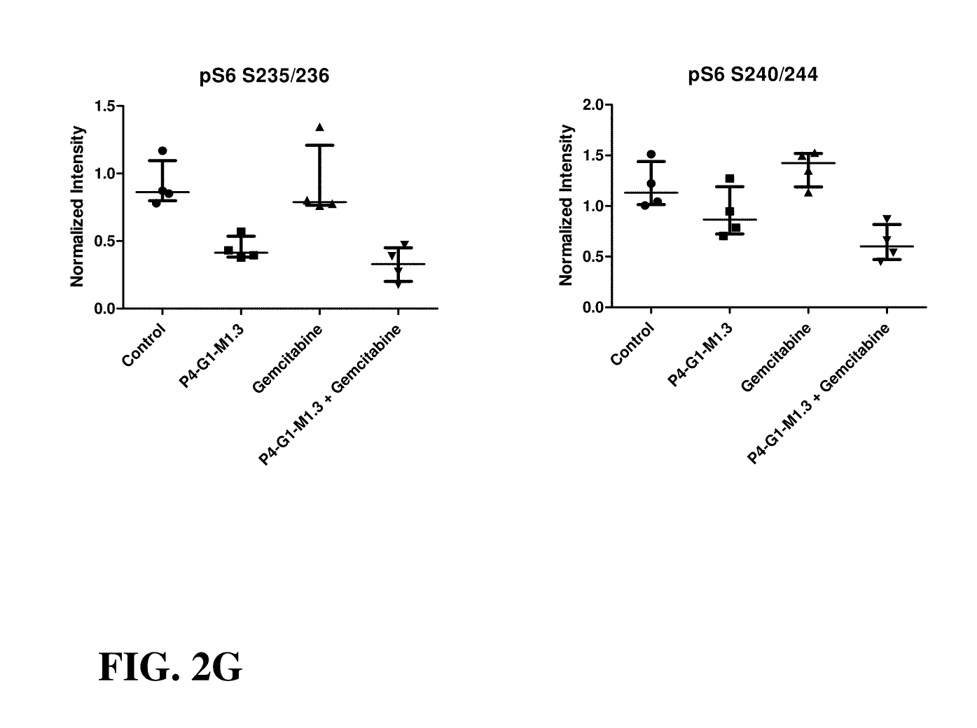

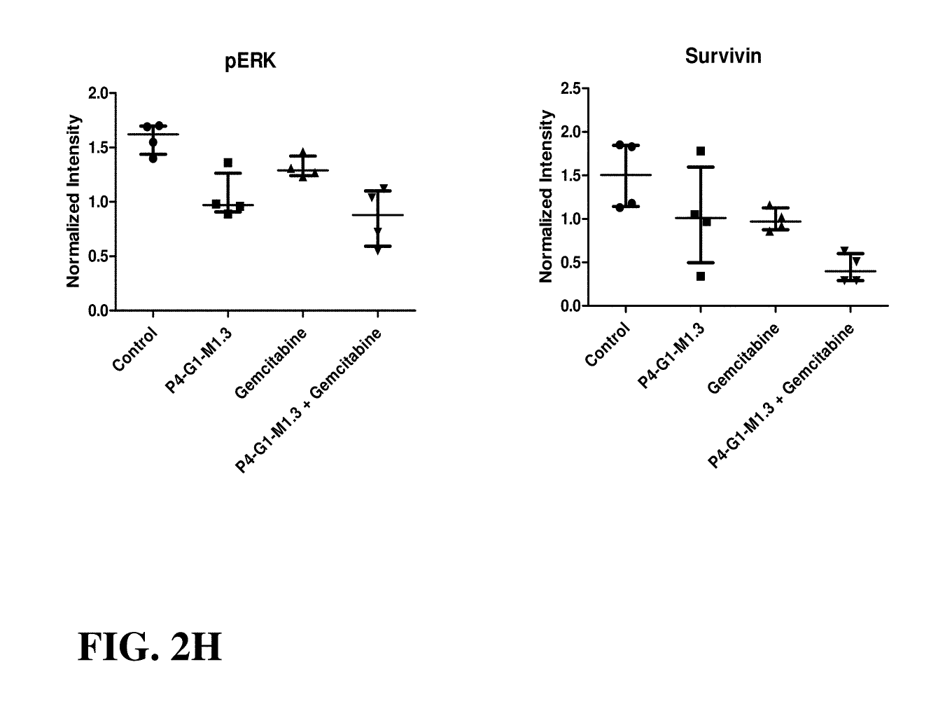

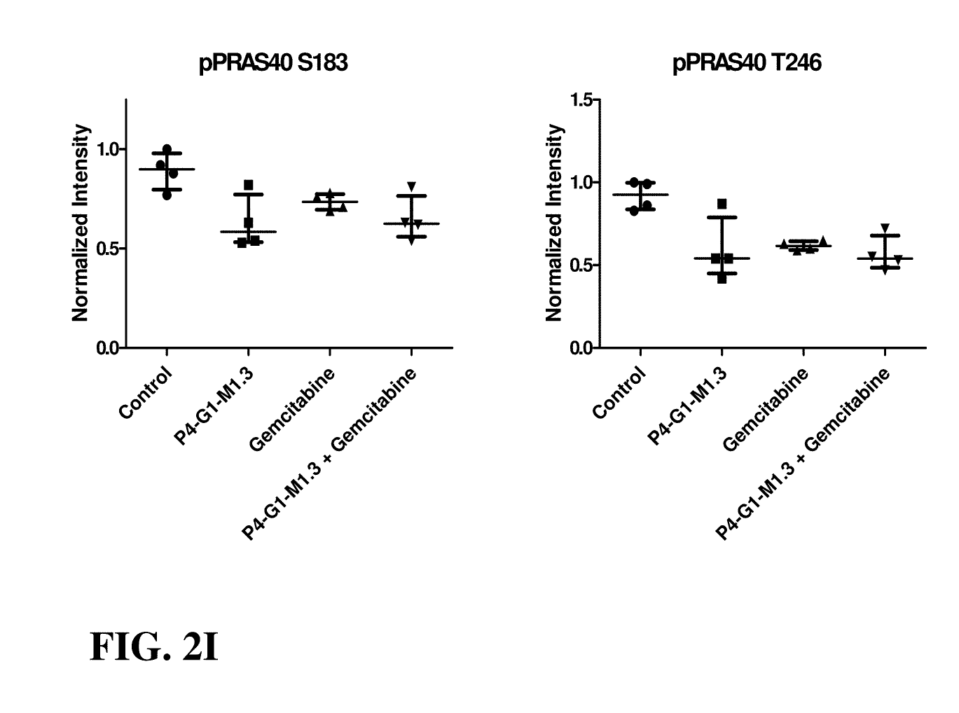

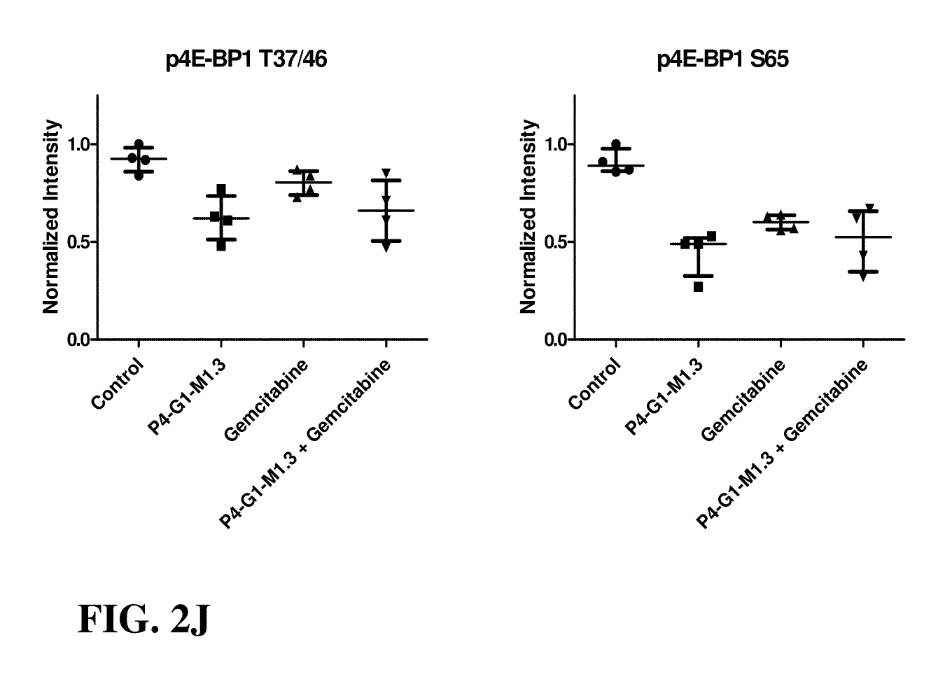

Preclinical xenograft data to support this Example were obtained using the pancreatic carcinoma cancer model BxPC-3. Using BxPC-3 xenografts, mice with control tumors were compared to those treated with monotherapy with P4-G1-M1.3, monotherapy with gemcitabine or combination therapy with P4-G1-M1.3 and gemcitabine. As shown in FIG. 2, P4-G1-M1.3 downregulates receptor complexes and inhibits PI3K/AKT/mTOR signaling in BxPC-3 PD study tumors. Results appear in the figures as follows: downregulation of IGF-1R and Insulin Receptor (FIG. 2A), EGFR and ErbB3 (FIG. 2B), ErbB2 (FIG. 2C), suppression of phosphoprotein in PI3K/AKT/mTOR signaling network such as phospho-AKT (FIG. 2D), phospho-FoxO and phospho-PDK1 (FIG. 2E), phospho-mTOR (FIG. 2F), phospho-S6(Figure 2G), pRAS40 (FIG. 2I) and p4EB-PB1 (FIG. 2J). In addition P4-G1-M1.3 inhibits ERK phosphorylation and potentiates apoptosis-inducing activity of gemcitabine (FIG. 2H)

### Example 20

Other tumor types that can be beneficially treated with effective amounts of bispecific anti-IGF-1R and anti-ErbB3 antibodies disclosed herein in combinations administered in accordance with this disclosure include thyroid carcinoma, head and neck squamous cell carcinoma, breast carcinoma, lung cancer (e.g., small-cell lung carcinoma, non-small-cell lung carcinoma), gastric carcinoma, gastrointestinal stromal tumors, ovarian carcinoma, bile duct carcinoma, endometrial carcinoma, prostate carcinoma, renal cell carcinoma, anaplastic large-cell lymphoma, leukemia (e.g., acute myeloid leukemia, T-cell leukemia, chronic lymphocytic leukemia), multiple myeloma, malignant mesothelioma, malignant melanoma, colon cancer, sarcoma. Each combination therapy will provide a combinatorially enhanced outcome.

### Example 21

Patients with pancreatic cancer (KRAS wild-type and KRAS mutant) are treated with a combination of an effective amount of a MEK inhibitor (e.g., GSK1120212, BAY 86-9766 or AZD6244) and an effective amount of P4-G1-M1.3. Patients are dosed with P4-G1-M1.3 as described in Example 1. The clinical dose of MEK inhibitor is the dose used for that inhibitor in phase II or phase III clinical trials. The combination therapy will provide a combinatorially enhanced outcome.

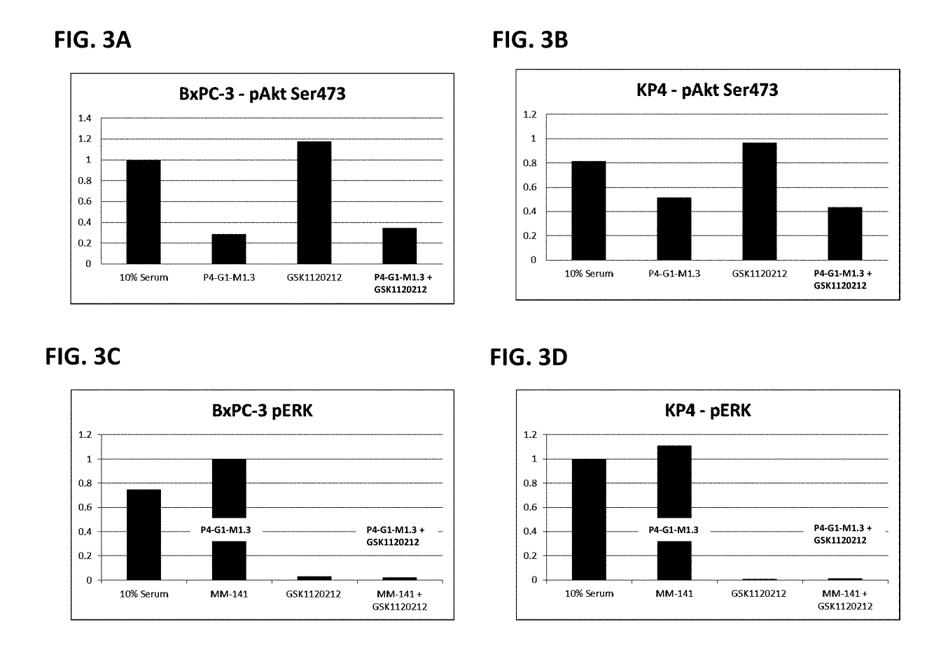

As shown in FIG. 3, treatment of cancer cells in vitro with GSK1120212 and P4-G1-M1.3 results in inhibition of signaling in both a wild-type and KRAS mutant background. The preclinical models to support this example are performed using BxPC-3 (KRAS wild-type) and KP4 (KRAS mutant) cell lines.

### Example 22

Women with locally advanced or metastatic breast cancer are treated with a combination of an effective amount of docetaxel (Taxotere®) and an effective amount of P4-G1-M1.3. Patients are dosed with P4-G1-M1.3 as described in Example 1. Patients are dosed with docetaxel at 25, 50, 75 or 100 mg/m² i.v. once every 3 weeks or per standard clinical practice. The combination therapy will provide a combinatorially enhanced outcome.

### Example 23

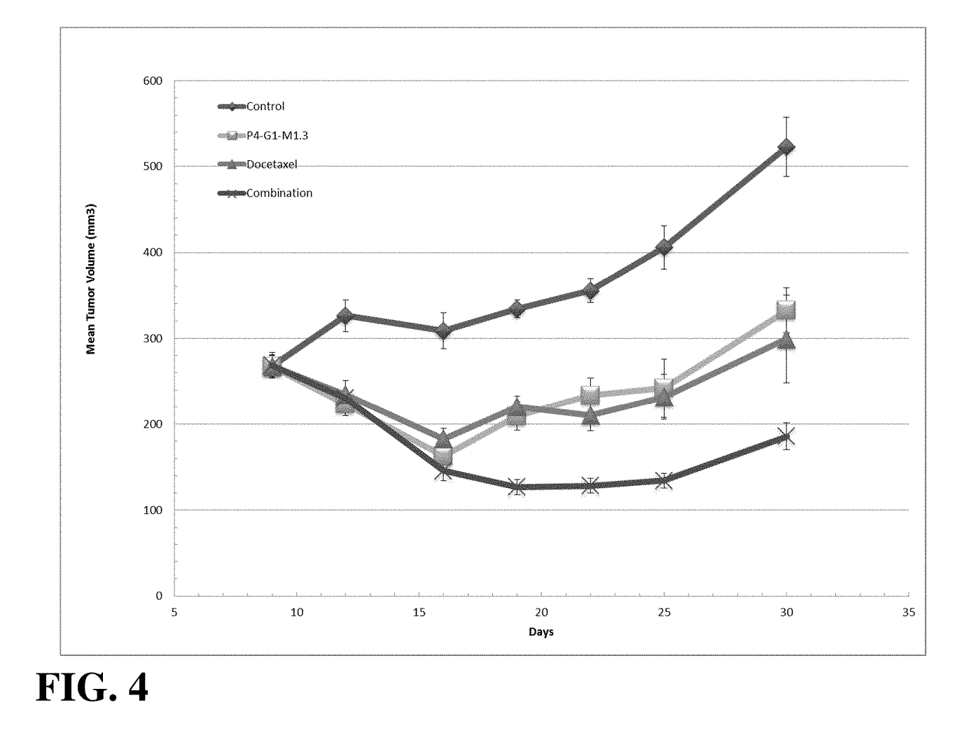

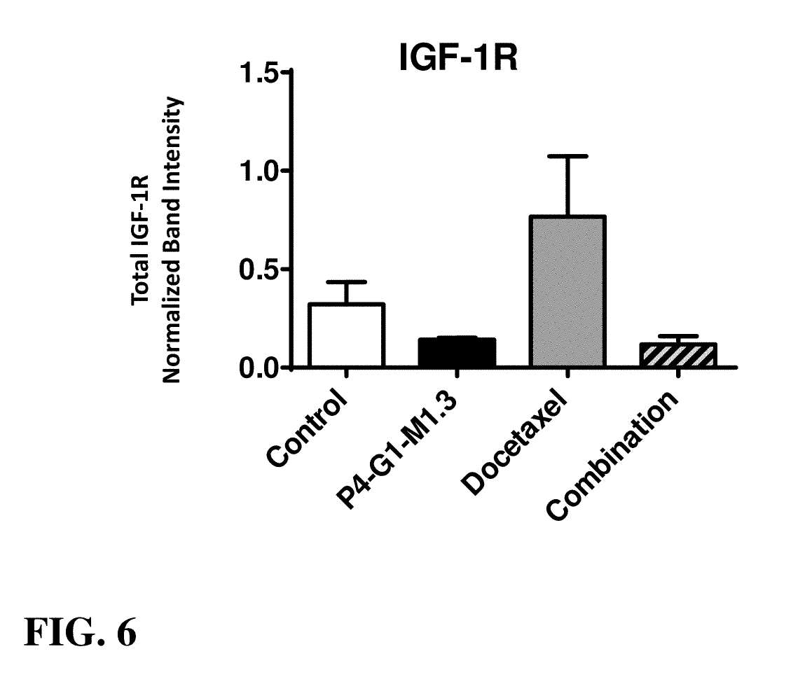

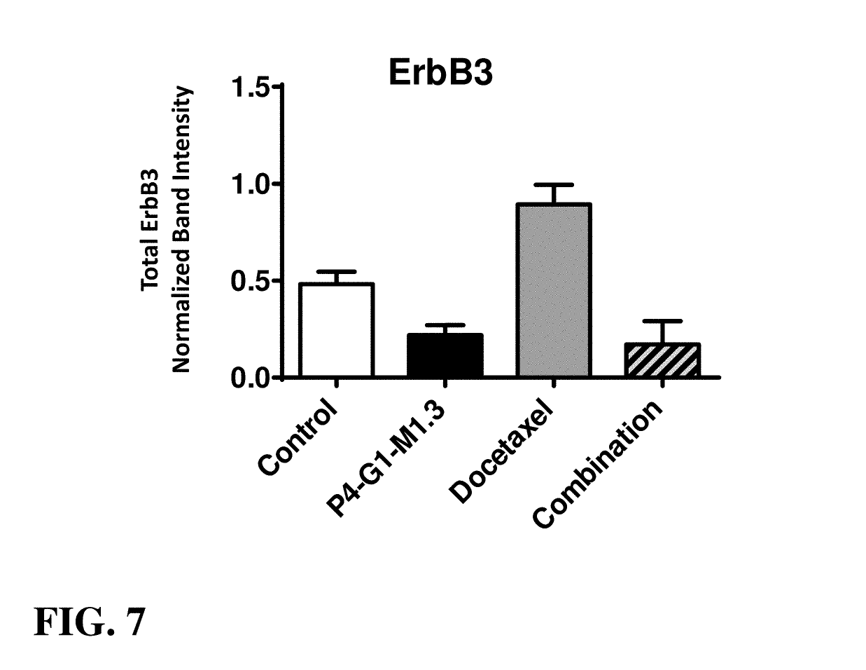

The advantages of combination therapy per Example 22 are demonstrated in a preclinical model in which the tumor type is a squamous cell carcinoma of the lung, prostate cancer or ovarian cancer, are demonstrated using the cell line DU145. As shown in FIG. 4, the combination of docetaxel and P4-G1-M1.3 results in inhibition of growth of DU145 prostate cancer cells in vivo. FIGS. 6 and 7 demonstrate the in vivo effects of P4-G1-M1.3 , Docetaxel, or the combination on total IGF-1R (FIG. 6) and total ErbB3 (FIG. 7) in DU145 xenografts. Statistical significance across groups was determined using the student's T-test (*,p<0.05 vs control.; #,p<0.05 vs docetaxel; a,p<0.05 vs P4-G1-M1.3).

### Example 24

Patients with metastatic breast cancer are treated with a combination of an effective amount of paclitaxel (Taxol®) or of eribulin and an effective amount of P4-G1-M1.3. Patients are dosed with P4-G1-M1 as described in Example 1. Patients are dosed per standard clinical practice for paclitaxel or eribulin. The combination therapy will provide a combinatorially enhanced outcome.

### Example 25

The treatments of Example 24 are repeated in patients where the tumor type is squamous cell carcinoma of the lung, prostate cancer or ovarian cancer. The results will be the same as obtained in Example 24.

### Example 26

Patients with hepatocellular carcinoma (HCC) are treated with P4-G1-M1.3 monotherapy. Patients are dosed with P4-G1-M1.3 as described in Example 1 (second line in patients refractory to sorafenib). Patients will obtain a statistically significant improvement in HCC symptoms (e.g., time to progression or progression-free survival at pre-defined intervals) compared to untreated historical controls or to best supportive care. Preclinical data to support this example may be obtained using HepG2 cells in vitro and in vivo.

### Example 27

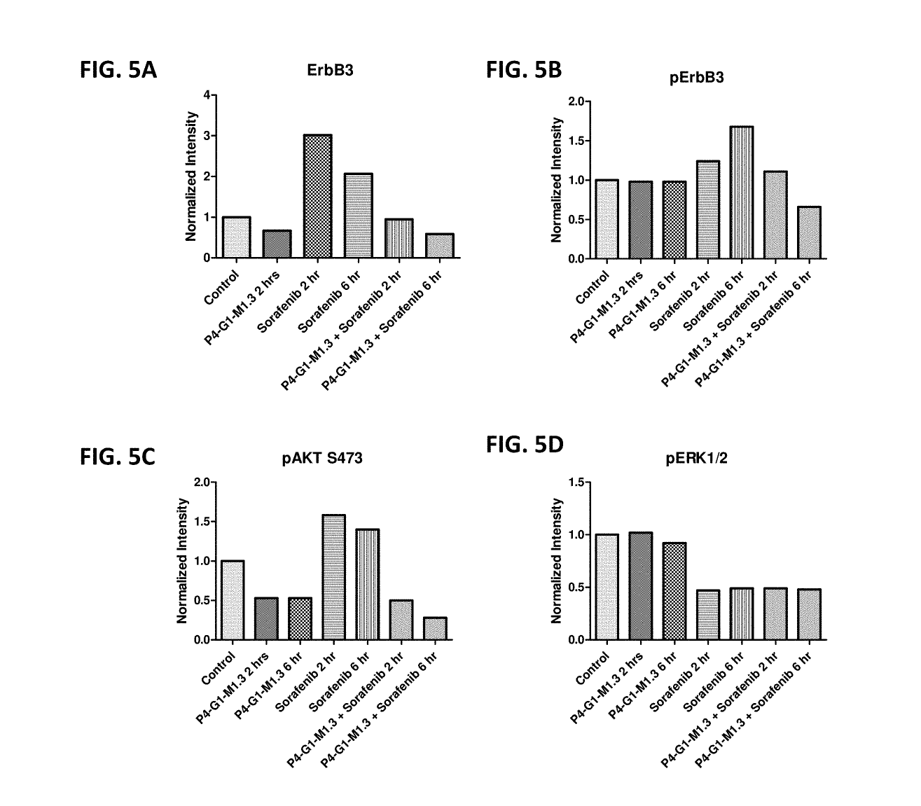

Patients with hepatocellular carcinoma (HCC) are treated with a combination of an effective amount of sorafenib and an effective amount of P4-G1-M1.3. Patients are dosed with P4-G1-M1.3 as described in Example 1. Patients are dosed with sorafenib at 400 mg daily. The combination therapy will provide a combinatorially enhanced outcome. Preclinical data to support this example may be obtained using HepG2 cells in vitro and in vivo. As shown in FIG. 5, treatment of HepG2 hepatocellular carcinoma cells with the combination of sorafenib and P4-G1-M1.3 results in downregulation of ErbB3 and inhibits downstream signaling when compared to treatments with sorafenib alone or with P4-G1-M1.3 alone.

### Example 28

Patients with melanoma are treated by administration of either monotherapy with an effective amount of vemurafenib (Zelboraf®) or an effective amount of P4-G1-M1.3, or with combination therapy comprising or consisting of administration of both the effective amount of vemurafenib or dabrafenib and the effective amount of P4-G1-M1.3. P4-G1-M1.3 is formulated and administered as described above in Example 1. Vemurafenib is given orally at 960 mg 1-2 times daily. Dabrafenib is administered as administered in dabrafenib phase III clinical trials. The combination therapy will provide a combinatorially enhanced outcome.

### Example 29

Patients with Ewing's sarcoma or metastatic pancreatic cancer are treated with a combination of an effective amount of irinotecan (Camptosar®) or nanoliposomal irinotecan and an effective amount of P4-G1-M1.3. P4-G1-M1.3 is formulated and administered as described above (e.g., Example 1). Nanoliposomal irinotecan is given i.v. at 80 mg/m² or 120 mg/m² q3w. Camptosar® is administered per manufacturer's directions. The combination therapy will provide a combinatorially enhanced outcome.

### EQUIVALENTS

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

### INCORPORATION BY REFERENCE

The disclosure of each and every U.S. and foreign patent and pending patent application and publication referred to herein is specifically incorporated by reference herein in its entirety.

1-69. (canceled)

70. A method for the treatment of pancreatic cancer in a human patient, the method comprising co-administering to the patient (i) an effective amount of a bispecific anti-IGF-1R and anti-ErbB3 antibody and (ii) an effective amount of an antimetabolite therapy, thereby treating the pancreatic cancer in the patient.

71. The method of claim 70, wherein the antimetabolite therapy is gemcitabine.

72. The method of claim 70, wherein the bispecific anti-IGF-1R and anti-ErbB3 antibody has an anti-IGF-1R module selected from the group consisting of SF, P4, M78, and M57.

73. The method of claim 70, wherein the bispecific anti-IGF-1R and anti-ErbB3 antibody has an anti-ErbB3 module selected from the group consisting of C8, P1, M1.3, M27, P6, and B69.

74. The method of claim 70, wherein the bispecific anti-IGF-1R and anti-ErbB3 antibody is P4-G1-M1.3.

75. The method of claim 70, wherein the bispecific anti-IGF-1R and anti-ErbB3 antibody is P4-G1-C8.

76. The method of claim 71, wherein the bispecific anti-IGF-1R and anti-ErbB3 antibody has an anti-IGF-1R module selected from the group consisting of SF, P4, M78, and M57.

77. The method of claim 71, wherein the bispecific anti-IGF-1R and anti-ErbB3 antibody has an anti-ErbB3 module selected from the group consisting of C8, P1, M1.3, M27, P6, and B69.

78. The method of claim 72, wherein the bispecific anti-IGF-1R and anti-ErbB3 antibody is P4-G1-M1.3.

79. The method of claim 72, wherein the bispecific anti-IGF-1R and anti-ErbB3 antibody is P4-G1-C8.

80. A method for the treatment of pancreatic cancer in a human patient, the method comprising co-administering to the patient (i) an effective amount of a bispecific anti-IGF-1R and anti-ErbB3 antibody and (ii) an effective amount of a taxane, thereby treating the pancreatic cancer in the patient.

81. The method of claim 80, wherein the taxane is nab-paclitaxel.

82. The method of claim 80, wherein the bispecific anti-IGF-1R and anti-ErbB3 antibody has an anti-IGF-1R module selected from the group consisting of SF, P4, M78, and M57.

83. The method of claim 80, wherein the bispecific anti-IGF-1R and anti-ErbB3 antibody has an anti-ErbB3 module selected from the group consisting of C8, P1, M1.3, M27, P6, and B69.

84. The method of claim 80, wherein the bispecific anti-IGF-1R and anti-ErbB3 antibody is P4-G1-M1.3.

85. The method of claim 80, wherein the bispecific anti-IGF-1R and anti-ErbB3 antibody is P4-G1-C8.

86. The method of claim 81, wherein the bispecific anti-IGF-1R and anti-ErbB3 antibody has an anti-IGF-1R module selected from the group consisting of SF, P4, M78, and M57.

87. The method of claim 81, wherein the bispecific anti-IGF-1R and anti-ErbB3 antibody has an anti-ErbB3 module selected from the group consisting of C8, P1, M1.3, M27, P6, and B69.

88. The method of claim 82, wherein the bispecific anti-IGF-1R and anti-ErbB3 antibody is P4-G1-M1.3.

89. The method of claim 82, wherein the bispecific anti-IGF-1R and anti-ErbB3 antibody is P4-G1-C8.
